(19) United States
(12) Patent Application Publication
(10) Pub. No.: US 2006/0154952 A1
(43) Pub. Date: Jul. 13, 2006

(54) Pharmaceutical compositions comprising a combination of rapamycin or its derivative and pimecrolimus for the treatment of inflammation-and ummunologically-mediated diseases

(76) Inventor: Henrietta Moore, Breitenfurt (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/547,185

(22) PCT Filed: Mar. 16, 2004
(86) PCT No.: PCT/EP04/02714

§ 371 Date: Sep. 19, 2005

(30) Foreign Application Priority Data
Mar. 17, 2003 (GB) 03060704
Mar. 25, 2003 (GB) 03068681
Aug. 15, 2003 (GB) 03192267

(51) Int. Cl. A61K 31/4745

(52) U.S. Cl. 514/291

(57) ABSTRACT

The present invention relates to pharmaceutical compositions comprising in combination rapamycin or a rapamycin derivative and a compound of formula I, e.g. useful for the treatment of inflammatory and immunologically-mediated diseases, including autoimmune diseases.

The present invention relates to pharmaceutical compositions, e.g. useful for the treatment of inflammatory and immunologically-mediated diseases, including autoimmune diseases.

In one aspect the present invention provides a pharmaceutical composition comprising in combination rapamycin or a rapamycin derivative and a compound of formula 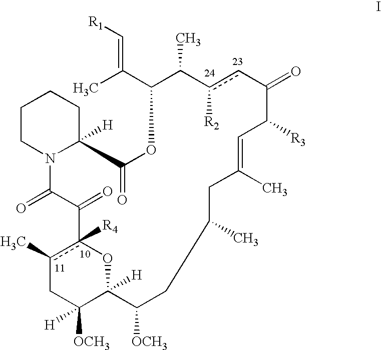
wherein either
R₁ is a group (a) of formula

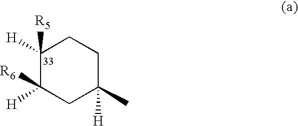

wherein
R₅ is chloro, bromo, iodo or azido,
R₆ is hydroxy or methoxy, and
R₄ is hydroxy and there is a single bond in 10,11 position; or absent, and there is a double bond in 10,11 position
   or
R₁ is a group (b) or (c) of formula 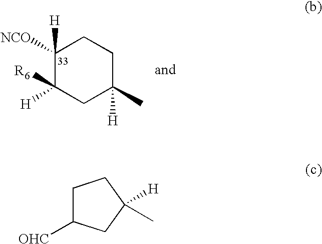
   wherein
   R₅ is as defined above, and
   R₄ is hydroxy and there is a single bond in 10,11 position,
R₂ is oxo and there is a single bond in 23,24 position; optionally protected hydroxy and there is a single or double bond in 23,24 position; or absent and there is a double bond in 23,24 position;
R₃ is methyl, ethyl, propyl or allyl
   beside at least one pharmaceutically acceptable excipient.

A pharmaceutical composition comprising in combination rapamycin or a rapamycin derivative and a compound of formula I is hereinafter designated as “a composition of (according to) the present invention”.

In another aspect the present invention provides a pharmaceutical composition of the present invention wherein a compound of formula I is a compound of formula 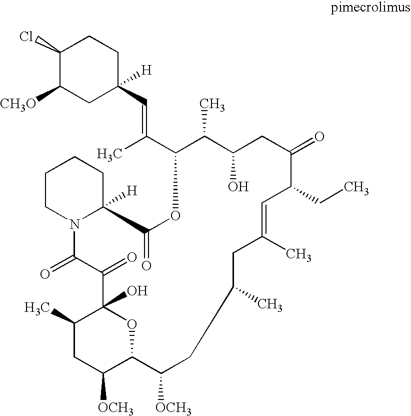
Pimecrolimus (INN recommended) (ASM981; Elidel™), i.e.

{[1E-(1R,3R,4S)]1R,9S,12S,13R,14S,17R,18E,21S,23S,24R,25S,27R}-12-[2-(4-chloro-3-methoxycyclohexyl)-1-methylvinyl]-17-ethyl-1,14-dihydroxy-23,25-dimethoxy-13,19,21,27-tetramethyl-1,28,dioxa-4-azatricyclo [22.3.1.0(4,9)]octacos-18-ene-2,3,10,16-tetraone is disclosed e.g. in EP 427 680 (33-epi-33-chloro-FR 520 of example 66a).

In another aspect the present invention provides a pharmaceutical composition of the present invention wherein the rapamycin derivative is a compound of formula 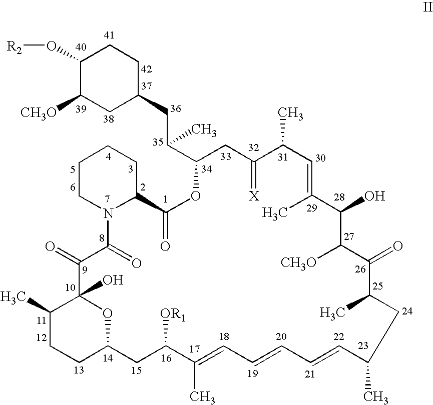
wherein
R₁ is CH₃ or (C₃₋₆)alkynyl,
R₂ is H, —CH₂—CH₂—OH, 3-hydroxy-2-(hydroxymethyl)-2-methyl-propanoyl or tetrazolyl, and
X is =O, (H,H) or (H,OH),
provided that R₂ is other than H when X is =O and R₁ is CH₃,
or a prodrug thereof when R₂ is —CH₂—CH₂—OH, e.g. a physiologically hydrolysable ether thereof.

Representative rapamycin derivatives of formula II include e.g. 32-hydrogenated rapamycin e.g. as described in WO 96/41807 and U.S. Pat. No. 5,256,790, incorporated herein by reference. such as e.g. 32-deoxorapamycin, 16-pent-2-ynyloxy-32-deoxorapamycin, 16-O-substituted rapamycin e.g. as disclosed in WO 94/02136, WO 95/16691 and WO 96/41807, the contents of which are incorporated herein by reference, such as e.g. 16-pent-2-ynyloxy-32(S or R)-dihydro-rapamycin, 16-pent-2-ynyloxy-32(S or R)-dihydro-40-O-(2-hydroxyethyl)-rapamycin, 40-O-substituted rapamycin e.g. as described in U.S. Pat. No. 5,258,389, WO 94/09010, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO 93/11130, WO 94/02136, WO 94/02485 and WO 95/14023, all of which are incorporated herein by reference, such as e.g. 40-[3-hydroxy-2-(hydroxymethyl)-2-methylpropanoate]-rapamycin (also designated as CCI779) or 40-epi-(tetrazolyl)-rapamycin (also designated ABT578). A preferred compound is e.g. 40-O-(2-hydroxyethyl)-rapamycin (Compound A hereinafter), e.g. disclosed in Example 8 in WO94/09010, or 32-deoxorapamycin, or 16-pent-2-ynyloxy-32(S)-dihydro-rapamycin, e.g. disclosed in WO96/41807. Rapamycin derivatives may also include (epi) rapalogs, e.g. as disclosed in WO98/02441 and WO01/14387, e.g. the compounds AP23573, AP23464, AP23675 or AP23841; or derivatives such as biolimus 7 or biolimus 9. Rapamycin derivatives may also include prodrugs of rapamycin such as TAFA93.

Suitable rapamycin derivatives are e.g. also described in U.S. Pat. No. 3,929,992 and U.S. Pat. No. 5,258,389.

Preferably the compositions of the present invention comprise rapamycin, also known as sirolimus (rapamycin; Rapamune^{R}) and/or the rapamycin derivative everolimus (Compound A, RAD001; 40-O-(2-hydroxyethyl)-rapamycin; Certican^{R}).

All patent literature cited herein is introduced herein by reference.

In another aspect the present invention provides a pharmaceutical composition comprising in combination 40-O-(2-hydroxyethyl)-rapamycin and pimecrolimus beside at least one pharmaceutically acceptable excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

A compound of a combination according to the present invention, i.e. a compound of formula I, e.g. pimecrolimus, may be in free form, in the form of a salt, in solvate form or in the form of a salt and a solvate, where salts and/or solvates exist.

In another aspect the present invention provides a composition of the present invention, wherein a compound of formula I is in the form of a salt.

A composition of the present invention may comprise one or more rapamycin derivatives, preferably rapamycin or one rapamycin derivative, and one or more compounds of formula I, preferably one, e.g. pimecrolimus.

A composition of the present invention is suitably based on emulsions, microemulsions, emulsion preconcentrates or microemulsion preconcentrates, or solid dispersions, especially water-in-oil microemulsion preconcentrates or oil-in-water microemulsions of rapamycin or a rapamycin derivative, e.g. a compound of formula II, and a compound of formula I, e.g. pimecrolimus.

A composition of the present invention may further comprise further pharmaceutically active compounds. Such further active compounds e.g. include anti-inflammatory and immunomodulatory agents.

In another aspect the pharmaceutical composition of the present invention comprises further an anti-inflammatory and/or an immunomodulatory agent.

A composition of the present invention includes
fixed combinations, in which a compound of formula I, e.g. pimecrolimus, and rapamycin or a rapamycin derivative, e.g. a compound of formula II, such as Compound A, are in the same formulation;
kits (=kit of parts), in which a compound of formula I, e.g. pimecrolimus, and rapamycin or a rapamycin derivative, e.g. a compound of formula II, such as Compound A, in separate formulations are sold in the same package, e.g. with instruction for co-administration; and
free combinations in which a compound of formula I, e.g. pimecrolimus, and rapamycin or a rapamycin derivative, e.g. a compound of formula II, such as Compound A, are packaged separately, but instruction for simultaneous or sequential administration are given.

In another aspect the present invention provides a pharmaceutical kit of parts comprising rapamycin or a rapamycin derivative, e.g. a rapamycin derivative of formula II, e.g. Compound A, and a compound of formula I, e.g. pimecrolimus, beside instructions for simultaneous or sequential administration.

We also have found surprisingly a pharmaceutical composition, e.g. solid, such as a tablet, comprising a fixed combination of rapamycin or a rapamycin derivative, including a compound of formula II, e.g. Compound A, and a compound of formula I, e.g. pimecrolimus, as active agents, e.g. an oral pharmaceutical composition.

In another aspect the present invention provides a pharmaceutical composition, e.g. an oral pharmaceutical composition as a fixed combination, e.g. a solid oral composition, in the form of a solid dispersion comprising rapamycin or a rapamycin derivative, e.g. Compound A, and a compound of formula I, e.g. pimecrolimus, and a carrier, e.g. cellulose, optionally in the presence of excipients as mentioned above.

Compositions in the form of a solid dispersion may be administered in any convenient form, e.g. tablet, capsule, granule or powder form.

Pharmaceutical compositions of the present invention, including an oral pharmaceutical composition, may be prepared as appropriate, e.g. according, such as analogously, to a method as conventional, or as described herein.

For the preparation of a fixed oral composition or combination, e.g. a tablet, of the present invention, a solid dispersion comprising a compound of formula I, e.g. pimecrolimus, and rapamycin or a rapamycin derivative, e.g. a compound of formula II, and a carrier, such as a cellulose, e.g. hydroxypropylmethylcellulose, may be used. Solid dispersions are known or may be obtained as appropriate. A tablet may be obtained e.g. by mixing the active agents with a carrier and optionally further excipient, dry granulation before or after adding further excipient, and compression of the mixture obtained. Further excipient includes e.g.
lubricants, such as Mg-stearate,
fillers, e.g. sugars, such as lactose,
flowing agents, such as siliciumdioxide, e.g. aerosils,
disintegrants, such as polyvinylpyrrollidones, e.g. povidones, crospovidones (homo- or co-polymers of N-vinyl-pyrrolidone).

The weight ratio of a compound of formula I, e.g. pimecrolimus, and rapamycin or a rapamycin derivative, e.g. a compound of formula II, in an oral composition of the present invention is not critical, e.g. including a weight ratio of from, e.g. about, 1:10 to 800:1. If a compound pimecrolimus and Compound A are used as active agents, pimecrolimus is preferably used in an excess, e.g. an appropriate weight ratio of pimecrolimus: Compound A includes a ratio of from, e.g. about, 10:1 to 800:1.

In another aspect the present invention provides a tablet for oral administration, comprising

Compound A and pimecrolimus as active agents, e.g. in the form of a solid dispersion,

a cellulose as a carrier, e.g. hydroxpropylmethylcellulose,

optionally disintegrants, e.g. a polyvinylpyrrolidone,

optionally flowing agents, e.g. an aerosil,

optionally lubricants, e.g. Mg-stearate,

optionally fillers, e.g. sugars such as lactose.

In another aspect the present invention provides a tablet of the present invention comprising in % by weight:

pimecrolimus: 2 to 8%, e.g. 4%;

Compound A: 0.05 to 0.25%, e.g. 0.13%;

hydroxypropylmethylcellulose (e.g. 3 cps): 10 to 30%, e.g. 21.4%;

lactose (e.g. 200 mesh): 0.6 to 2.0%, e.g. 1.2%;

butylated hydroxytoluene: 0.005 to 0.025%, e.g. 0.013%

lactose spray dried: 25.0 to 70.0%, e.g. 50.8%;

crospovidone: 10.0 to 40.0%, e.g. 20.0%;

silicium dioxide colloidal (Aerosil 200®): 0.5 to 2.5%; e.g. 1.5%

magnesium stearate: 0.5 to 2.0%, e.g. 1.0%.

Such tablet may e.g. contain, e.g. about, 10 to 60 mg of pimecrolimus, e.g. 30 mg, and 0.1 to 2 mg of Compound A, e.g. 1 mg.

In another aspect the present invention provides the use of a combination of rapamycin or a rapamycin derivative, including a compound of formula II, and a compound of formula I, e.g. pimecrolimus, as a pharmaceutical.

In another aspect the present invention provides the use of a combination of rapamycin or a rapamycin derivative, such as a compound of formula II, and a compound of formula I, e.g. pimecrolimus, for the manufacture of a medicament for the treatment of inflammatory bowel disease and diseases associated therewith.

IBD in man, like many other chronic inflammatory or autoimmune diseases, can be seen as a result of a convergence of interactions between susceptibility genes, the environment and the immune system. The extent of interplay between these factors will determine the clinical manifestation of disease; Crohn's disease (CD) or ulcerative colitis (UC) and the various clinical subgroups as defined by severity and location. The leading current hypothesis on the pathogenesis of IBD is that it is a dysregulated immune response to normal enteric bacterial antigens (see e.g. Kirsner J B, Inflammatory Bowel Disease. 5th ed; W. B. Saunders, p. 208-39, p. 299-304; p. 305-14 and p. 315-25). In both CD and UC the number of lamina propria T lymphocytes is increased (see e.g. Selby et al, (1984), Gut; 25 (1):32-40). There is evidence, mostly from experimental models such as the SCID-IBD model, that suppressor subsets of T cells prevent a reaction to the normal flora encountered in the gut (see e.g. Groux H, et al, (1999), Immunol. Today; 20 (10):442-5). In IBD this balance has been disturbed and the ensuing inflammatory cascade develops into a chronic response. The transfer of syngeneic CD4⁺ T cells which express high levels of CD45RB (CD4⁺CD45RB^{HI}) to SCID mice (SCID-IBD) induces a severe colitis and wasting disease which usually results in death of the mice within 4-8 weeks following transfer of the pathological CD4⁺CD45RB^{HI} T cells (see e.g. Powrie F et al (1993), Int. Immunol.; 5 (11):1461-71). This severe model of colitis shares many of the features of human IBD and has indeed been used throughout the 1990's to enhance further understanding of the disease in man and is regarded to reflect IBD more than any other model. These include colitis induced by administration of exogenous agents or by gene manipulation (see e.g. Blumberg R S et al, (1999) Curr. Opin. Immunol.; 11 (6):648-56; Strober W et al, (1998), Ann. Intern. Med.; 128 (10):848-56; Strober W et al, (1998), Scand. J. Immunol.; 48 (5):453-8). Using the SCID-IBD model, antibodies against TNFα, IFNγ and IL-12 have shown transient and partial beneficial effects (see e.g. Powrie F et al (1994), Immunity.; 1 (7):553-62; Simpson S J, et al (1998), J. Exp. Med.; 187 (8):1225-34; Mackay F et al (1998), Gastroenterology; 115 (6):1464-75). More recently, beneficial effects have also been reported with antibodies to the CD134 ligand (to OX40L, see e.g. Malmstrom V et al (2001), J. Immunol.; 166 (11):6972-81); to CD154 (see e.g. Liu Z. et al, (2000), J. Immunol.; 164 (11):6005-14; De Jong Y P et al (2000), Gastroenterology; 119 (3):715-23) and to the β7 integrin or MAdCAM-1 (see e.g. Picarella D et al, (1997), J. Immunol.; 158 (5):2099-106). Together these data demonstrate that the disease can be abrogated by inhibition of either the main inflammatory mediators including TNFα, and/or cytokines involved in neutralizing Th1 type differentiation and effector function or by interfering with the antigen presentation steps occurring in the lymph nodes or with the process of leucocyte homing of these cells.

In another aspect the present invention provides the use of a combination of rapamycin or a rapamycin derivative, such as a compound of formula II, e.g. Compound A, and a compound of formula I, e.g. pimecrolimus, for the manufacture of a medicament for the treatment of a disease selected from the group consisting of atopic dermatitis, contact dermatitis and further eczematous dermatoses, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias and acne.

E.g., in view of the results obtained, a combination or composition of the present invention is indicated for use as a pharmaceutical on diseases wherein a compound of formula I, including pimecrolimus, and/or rapamycin or a rapamycin derivative is pharmaceutically active, e.g. including inflammatory and immunologically-mediated diseases, including autoimmune diseases, e.g. allergic conditions, such as atopic dermatitis, contact dermatitis and further eczematous dermatoses, seborrhoeic dermatitis, contact hypersensitivity, lichen planus, Pemphigus, bullous Pemphigoid, Epidermolysis bullosa, urticaria, angiodemas, vasculitides, erythemas, cutaneous eosinophilias, acne, asthma; psoriasis, systemic lupus erythematosus, rheumatoide arthritis, inflammatory bowel diseases (IBD), multiple sclerosis, insulin-dependent diabetes, Sjögren's syndrome, endogenous posterior uveitides, Hashimoto's thyroiditis, vasculitides; or to prevent rejections of allografts, e.g. including renal or hepatic transplants; or graft vs host diseases, preferably IBD and diseases associated with IBD.

In another aspect the present invention provides a method for treatment of a disease selected from the group consisting of atopic dermatitis, contact dermatitis and further eczematous dermatoses, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias and acne comprising administering to a subject in need of such treatment an effective amount of a composition of the present invention or a pharmaceutical kit-of-parts of the present invention.

In another aspect the present invention provides a method of treatment of inflammatory bowel disease and diseases associated therewith comprising administering to a subject in need of such treatment an effective amount of a composition of the present invention or a pharmaceutical kit-of-parts of the present invention.

A composition of the present invention may be administered by any appropriate route, e.g. analogously to administration of a compound of formula I, e.g. pimecrolimus and rapamycin or a rapamycin derivative, e.g. a compound of formula II.

The compositions of the present invention may be prepared as appropriate, e.g. analogously to a method as conventional, e.g. by mixing a compound of formula I, e.g. pimecrolimus, and rapamycin or a rapamycin derivative, in combination or each separately, beside at least one pharmaceutically acceptable excipient.

We also have found that a compound class including FK506 in combination with rapamycin or a rapamycin derivative is useful in the treatment of inflammatory and immunologically-mediated diseases.

In another aspect the present invention provides the use of a combination of rapamycin or a rapamycin derivative, e.g. a rapamycin derivative of formula II, e.g. Compound A, and a compound of formula 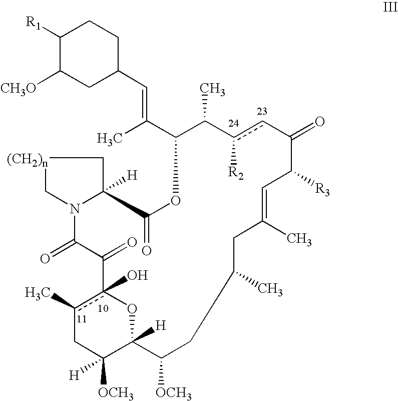
wherein
R₁ is hydroxy or protected hydroxy,
R₂ is hydrogen, hydroxyl or protected hydroxyl,
R₃ is methyl, ethyl, propyl or allyl,
n is an integer of 1 or 2, and
the symbol of a line and dotted line is a single bond for the manufacture of a medicament for the treatment of a disease selected from the group consisting of atopic dermatitis, contact dermatitis and further eczematous dermatoses, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias and acne.

In another aspect the present invention provides the use of a combination of rapamycin or a rapamycin derivative, e.g. a rapamycin derivative of formula II, e.g. Compound A, and a compound of formula III for the manufacture of a medicament for the treatment of inflammatory bowel disease.

In another aspect the present invention provides the use of a combination of rapamycin or a rapamycin derivative, e.g. a rapamycin derivative of formula II, e.g. Compound A, and a compound of formula III, wherein a compound of formula III is a compound of formula

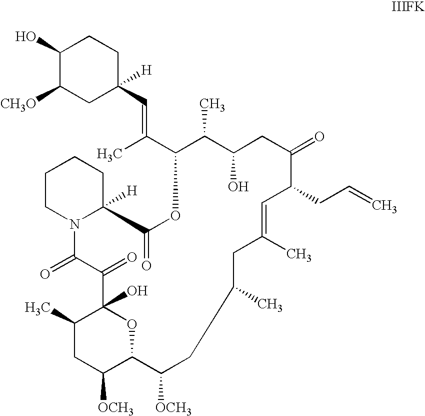

We have also found that a compound of formula II is useful in the treatment of various skin related diseases, such as atopic dermatitis, contact dermatitis and further eczematous dermatitises, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias and acne.

Accordingly, in another aspect the present invention provides a method for treating atopic dermatitis, contact dermatitis and further eczematous dermatitises, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias, and acne in a subject in need thereof, comprising administering to said subject a therapeutically effective amount of a compound of formula 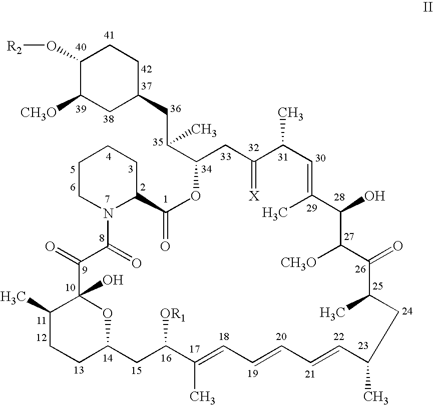
wherein
R₁ is CH₃ or (C₃₋₆)alkynyl,
R₂ is H, —CH₂—CH₂—OH, 3-hydroxy-2-(hydroxymethyl)-2-methyl-propanoyl or tetrazolyl, and
X is =O, (H,H) or (H,OH),
provided that R₂ is other than H when X is =O and R₁ is CH₃,
or a prodrug thereof when R₂ is —CH₂—CH₂—OH, e.g. a physiologically hydrolysable ether thereof.

Particularly preferred rapamycin derivatives of formula II for this aspect of the invention are 40-O-(2-hydroxyethyl)-rapamycin (Compound A hereinafter), 40-[3-hydroxy-2-(hydroxymethyl)-2-methylpropanoate]-rapamycin (also called CCI779), 40-epi-(tetrazolyl)-rapamycin (also called ABT578), 32-deoxorapamycin, 16-pent-2-ynyloxy-32(S)-dihydro rapamycin, or TAFA-93. Even more preferred is Compound A.

The present invention further provides:
1. A rapamycin derivative of formula II as described above, e.g. Compound A, for use in the method of treating skin-related diseases as defined above.
2. A rapamycin derivative of formula II as described above, e.g. Compound A, for use in the preparation of a pharmaceutical composition for use in the method of treating skin-related diseases as defined above.
3. A pharmaceutical composition for use in the method of treating skin-related diseases as defined above, comprising a rapamycin derivative of formula II as described above, e.g. Compound A, together with one or more pharmaceutically acceptable diluents or carriers therefore.

Utility of rapamycin derivatives of formula II and of combinations of the invention as described above in treating diseases and conditions as hereinabove specified, may be demonstrated in standard animal or clinical tests, e.g. as described hereinafter.

Daily dosages required in practicing the methods of the present invention will vary depending upon, for example, the chemical nature and the pharmacokinetic data of a compound of the present invention employed, the individual host, the mode of administration and the nature and severity of the conditions being treated. However, in general, for satisfactory results in larger mammals, for example humans, an indicated daily dosage is in a similar, but lower range, than the range in which a compound of formula I, e.g. pimecrolimus, and rapamycin or a rapamycin derivative are generally administered.

A preferred daily dosage range is about from 0.1 to 25 mg of rapamycin or a rapamycin derivative of formula II, e.g. Compound A, as a single dose or in divided doses. Suitable daily dosages for patients are on the order of from e.g. 0.1 to 25 mg p.o. of rapamycin or a rapamycin derivative of formula II, e.g. Compound A.

A composition of the present invention may be administered by any appropriate route, e.g. analogously to administration of a compound of formula I, e.g. pimecrolimus and rapamycin or a rapamycin derivative, e.g. a compound of formula II. For example, rapamycin or a rapamycin derivative of formula II, e.g. Compound A, may be administered by any conventional route, in particular enterally, e.g. orally, e.g. in the form of tablets, capsules, drink solutions, nasally, pulmonary (by inhalation) or parenterally, e.g. in the form of injectable solutions or suspensions. Suitable unit dosage forms for oral administration comprise from ca. 0.05 to 12.5 mg, usually 0.25 to 10 mg of rapamycin or a rapamycin derivative of formula II, e.g. Compound A, together with one or more pharmaceutically acceptable diluents or carriers therefore.

In the present description the terms “treatment” or “treat” refer to both prophylactic or preventive treatment as well as curative or disease modifying treatment, including treatment of patients at risk of contracting disease or suspected to have contracted disease as well as patients who are ill or have been diagnosed as suffering from a disease or medical condition.

### DESCRIPTION OF THE FIGURES

FIG. 1 to FIG. 3

Show the body weight of the SCID-IBD mice after having been reconstituted with 2×10⁵ CD4⁺CD45RB^{HI} T cells, treated and untreated with pimecrolimus (ASM), Compound A and a combination of ASM+Compound A. PBS-mice are non-transferred mice. Treatment started on day 1 after transfer and continued daily. Data are expressed as the mean % body weight (relative to weight on day 0)±s.e.m. for n=8 (with few exceptions where n=5, 6 or 7) mice per group **p<0.01, *p<0.05 relative to the vehicle-treated group the loss of body weight is shown.

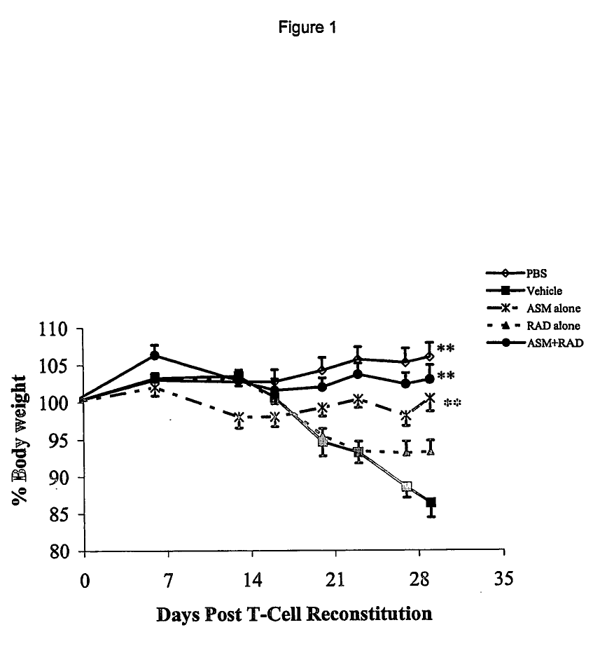

In FIG. 1 the results from using a Dosage 1 (Compound A: 0.1 mg/kg/d, ASM: 60 mg/kg/d),

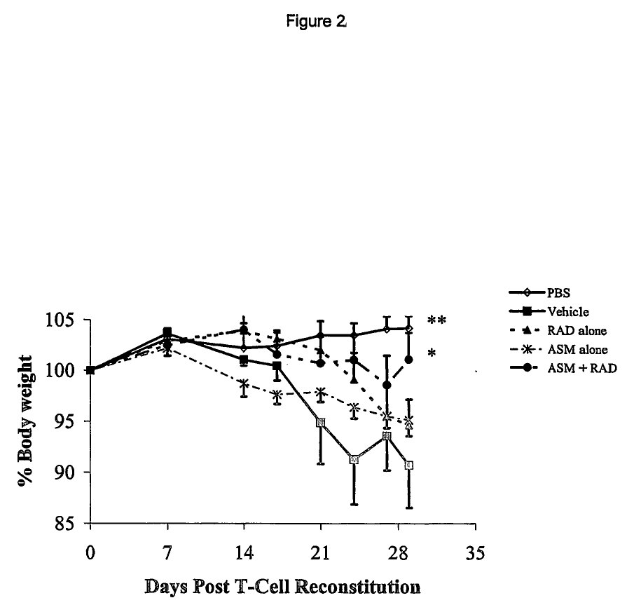

in FIG. 2 the results from using a Dosage 2 (Compound A: 0.1 mg/kg/d, ASM: 30 mg/kg/d, and

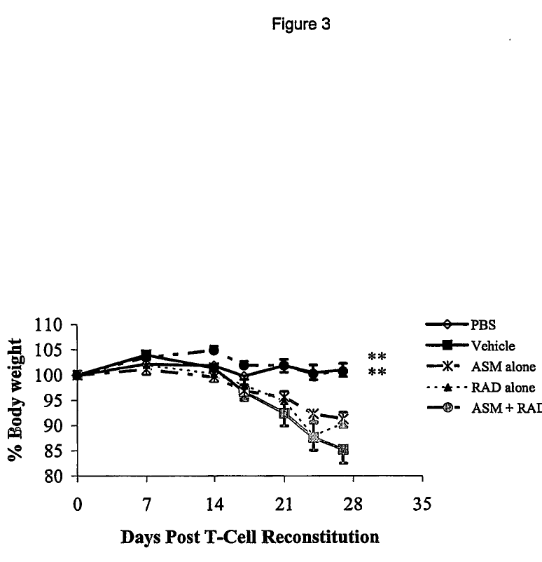

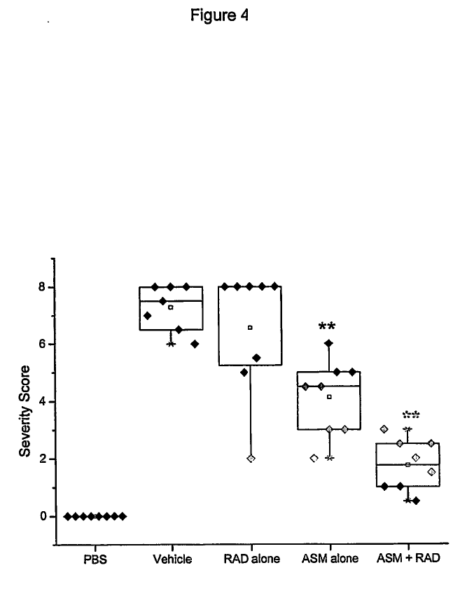

in FIG. 3 the results from using a Dosage 3 (Compound A: 0.05 mg/kg/d, ASM: 10 mg/kg/d) are shown.

FIG. 4

shows the severity of colon inflammation in SCIB-IBD mice after various treatments, severity scores are given as described in example 1.

Temperatures are given in degree Celsius (°) and are uncorrected

The following abbreviations are used herein:
ASM pimecrolimus
AUC Area under the Curve
ANOVA Analysis of variance
BW Body Weight
CD Crohn's disease
CyA Cyclosporin A
FACS Fluorescence Activated Cell Sorter
H & E Haematoxylin & eosin
HTAB Hexadecyltrimethylammoniumbromide
IBD Inflammatory Bowel Disease
IFN Interferon
IL- Interleukin
i.p. Intra-peritoneal
MPO Myeloperoxidase
MLN Mesenteric lymph node
MTOR mammalian Target of Rapamycin
PBS Phosphate buffered saline
PBS-(mice) SCID mice receiving PBS i.p. instead of disease-causing T cells
p.o. Per os
RAD Compound A, everolimus
SCID Severe Combined ImmunoDeficient
s.e.m. Standard error of the mean
SD Standard deviation
TMB 3,3′,5,5′-Tetramethylbenzidine
TNF Tumour necrosis factor
UC Ulcerative colitis

### Example 1

Test Methods

SCID-IBD Mouse Model

Female BALB/cJ and C.B.17 scid/scid mice (Fox Chase SCID®, Bomholtgaard, Denmark) are maintained in ventilated cage racks (Micro-vent type 84-II, Allentown Caging Equipment Co., Allentown, N.J., USA) under specific pathogen free conditions. The experimental procedures performed on the mice are authorized under the license number MA 1048/00 (Magistratabteilung No. 58, Amt der Wiener Landesregierung).

Briefly, CD4⁺CD45RB^{HI} T lymphocytes are isolated from BALB/c mouse spleens by two-colour FACS-sorting through a live/dead gate and injected (2×10⁵ cells/mouse, i.p.) into 6-9 week old female SCID mice. Negative control mice receive PBS i.p. and one such mouse is in each cage as a sentinel to monitor possible infections in this immunodeficient colony (PBS-mice).

SCID mice are first reconstituted with the CD4⁺CD45RB^{HI} T cells and then treated with test compounds or vehicle as controls. As test compounds Compound A (RAD) and pimecrolimus (ASM) are used. 3 separate studies using different dosaging are carried out. In each study both compounds are administered alone and combined. All 3 studies follow the same basic protocol of 4 groups of mice with one group receiving, by daily oral gavage, both vehicles (placebo), one group Compound A alone, one group ASM alone, and one group a combination of Compound A and pimecrolimus, from day 1 to day 29 after cell transfer.

Dosages Administered:

| | |
|---|---|
| Study 1: Dosage 1: | Compound A: 0.1 mg/kg/d, ASM: 60 mg/kg/d |
| Study 2: Dosage 2: | Compound A: 0.1 mg/kg/d, ASM: 30 mg/kg/d, and |
| Study 3: Dosage 3: | Compound A: 0.05 mg/kg/d, ASM: 10 mg/kg/d. |

The body weight of each mouse is monitored throughout and at the end of the study mice are weighed.

The mice are anaesthetized at the end of the study and blood withdrawn via cardiac puncture for separation into serum which is frozen and stored at −80°.

Blood is also taken into 10 mM EDTA for preparation for FACS analysis.

The spleen and MLN are taken from each mouse, weighed and processed for FACS analysis of single cell suspensions.

A photographic picture is taken of the opened abdominal cavity and then a 1 cm long piece of the colon, just above the rectum, is fixed in formalin for histology.

Specimens are embedded in paraffin, sections (3 μm thick) are cut and stained with H & E. Four colour FACS analysis is performed on cell suspensions prepared from the blood, spleen and mesenteric lymph nodes (MLNS) of each mouse.

Antibodies are obtained from Pharmingen. Cells which are double positive for CD3 and CD4 are counted as lymphocytes. Cells positive for Ly-6G (myeloid differentiation antigen) and negative for CD3 or CD4 are neutrophils (also their cellular characteristics (forward and side scatter) distinguishes them from the other cells).

The number of cells per sample is calculated from the total cell number in each cell suspension and the % of positively identified (samples analysed on a FACS Calibur® using CellQuest Pro® software) cells per sample.

The mean of these values for a group of mice can thus be calculated. To process some of the data further, the determined values are adjusted by subtracting the background value found in non-transferred mice and calculating the % reduction, or indeed increase, in mean cell number, relative to the placebo treated group.

All data presented as mean values are qualified by an s.e.m. value. The body weight data are analysed either by repeated measures analysis or by first finding the mean AUC and then applying an ANOVA multiple comparison test with Tukey post hoc correction. Histology scores are represented as the mean severity score and compared with a Wilcoxon-Mann-Whitney test with exact p-values (StatXact-5 software) and Bonferroni post hoc correction factor. All other data are first analysed for normal distribution and then the ANOVA test for multiple comparison is applied. Statistical significance is taken as p<0.05.

The developing reaction in the SCID mouse to the transfer of CD4⁺CD45RB^{HI} T cells shows that T cell expansion is the first event and the inflammatory sequelae follow, which can be measured as a critical loss of body weight, a severe colon inflammation and systemic signs such as increased serum haptoglobin levels and neutrophilia. The increasing T cell population with time also results in an increase in size of lymphoid organs which are otherwise comparatively small in the non-transferred SCID mouse. By day 28 post T cell transfer the inflammation is relatively consistent within a group of mice and this has been adopted as the shortest time where accurate effects of treatment with test compounds can be measured. The effects of a range of doses of Compound A and ASM as single preventative compounds are, in brief, comparative effective dose ranges are found to be from 30 to 100 mg/kg/d administration of ASM and 0.1 to 1 mg/kg/d of Compound A. In the present tests sub-maximal doses of each compounds are used and the effect of the single and combined treatment in the same study is determined.

Results

Synergistic Effect of Combined Treatment of SCID-IBD Mice with Compound A and ASM on the Loss of Body Weight

The first visible symptom resulting from the transfer of disease causing CD4⁺CD45RB^{HI} T cells to SCID mice is a severe loss of body weight, usually significant 21 days after transfer. These mice (designated as placebo in these studies, e.g. in the Figures) continue to lose body weight until death occurs, generally 4 to 8 weeks after cell transfer. Non-transferred mice (designated as “PBS(-mouse)” in the Figures) remain stable or gain weight in the course of the study. In the 3 individual studies described herein, the mean % difference in body weight between the non-transferred and the vehicle-treated mice is calculated to be 16.3±1.8%.

If transferred SCID mice are treated with Compound A alone or with ASM alone, a significant, but low inhibition of weight loss is only found for mice treated with ASM at a dosage of 60 mg/kg/d. Whereas thus dosages used of either compound alone have only minor effects on the loss of body weight, a combined administration of Compound A and ASM has a significant protective effect, even at the lowest doses of ASM (10 mg/kg/d) and Compound A (0.05 mg/kg/d), see e.g. FIG. 3. Since the dosage administered of Compound A and ASM alone have no or only a small significant effect on weight loss, the protective effect of a combined administration cannot be additive, but is synergistic.

Further data (not shown) has been obtained which confirms the synergistic effect of a combination according to the present invention.

In short, we have found a synergistic effect of a combination of ASM and Compound A in the SCID-IBD model, when using sub-maximal doses of 60,30 and 10 mg/kg/d of ASM and 0.1, 0.1 and 0.05 mg/kg/d of Compound A, respectively. The dose of 60 mg/kg/d of ASM alone still elicited significant effects on its own but all other single treatment groups resulted in only mild disease inhibition.

Specifically we have obtained the following results:
All three combinations, most strikingly also the lowest doses of 10 mg/kg/d of ASM and 0.05 mg/kg/d of Compound A, results in no loss of body weight (data shown, see FIGS. 1 to 3)
At the low dose combinations of 10 and 0.05 mg/kg/d a synergistic effect on the colon inflammation is shown. Either compound alone had very little effect, whilst the severity scores in the combined treatment group are mostly mild (data not shown).
ASM potently reduces serum haptoglobin levels at 60 and 30 mg/kg/d whilst Compound A is very weakly active at 0.1 or 0.05 mg/kg/d. Again, synergism is evident in the low dose study where the % reduction of each compound alone was 32.7% (ASM 10 mg/kg/d) or 27.4% (Compound A, 0.05 mg/kg/d) whereas the combined treatment group is significantly inhibited by 81.0% (data not shown).
In the low dose study there is no significant reduction in lymphocyte numbers in any of the treatment groups, although the numbers in the combined group are reduced (46.5, 19.0 and 48.0% in blood, spleen and MLN, respectively) relative to vehicle-treated mice. In the study where ASM and Compound A are given at higher doses (30 mg/kg/d and 0.1 mg/kg/d, respectively; study 2) there are significant reductions in lymphocyte numbers (73.0, 80.3 and 90.7% in blood, spleen and MLN, respectively) whereas numbers in the single treatment groups (ASM or Compound A, respectively, administered alone) are not reduced. Similarly, with the higher dose of ASM (60 mg/kg/d; study 1), which alone decreases lymphocyte numbers specifically in the MLN (73.5%), the combination with Compound A (0.1 mg/kg/d) results in significant reductions in both blood (65.4%) and spleen (64.8%) in addition to an enhanced reduction (93.2%) in the MLN. The dose of 0.1 mg/kgid of Compound A alone has only weal, if any effects on lymphocyte numbers.
   ASM981 Combined with Compound A Acts Synergistically to Reduce Colon Inflammation in SCID-IBD Mice

The inflammation in the colon of SCID-IBD mice shares many features observed in lesional tissue of a Crohn's diseased colon. In order to examine the intestinal mucosal damage in SCID-IBD mice a histological examination of the colon sections was performed according to defined morphological parameters. The severe colitis associated with this model of IBD is characterized with a massive cellular infiltrate which in places involves nearly all of the submucosa. The infiltrate is mixed and comprised of lymphocytes, macrophages, neutrophils and some eosinophils. Also evident is the loss of crypt architecture, extensive elongation and epithelial hyperplasia. A severely inflamed colon section will also have crypt abscesses. Numeric scores were given as per the following scheme: a score of 0 to 3 is given for the leucocyte infiltrate where 1 is mild, 2 is moderate and 3 is a severe infiltrate including the submucosal layer. A score of 0 to 2 is given for Goblet cell depletion where 2 is given for a complete lack of the alcian blue staining, 0 is given for a normal amount of staining and 1 is intermediate. A score of 0 to 3 is given for mucosal hyperplasia and degenerative changes which include crypt elongation, epithelial hyperplasia, presence of crypt abscesses, erosions and epithelial necrosis. The maximum possible score is 8 and (PBS)-mice are scored 0. We find that the dose of 60 mg/kg/d ASM981 alone has a significant effect. Doses of Compound A below 0.5 mg/kg/d are not significantly active and also not in this model. However, the two compounds together reduce the inflammation (FIG. 5) to mild levels (scores below 4) that could not be achieved by the additive effects of each alone. In conclusion, in the SCID-IBD model, at dosages which alone have minimal effects, combined treatment with pimecrolimus and Compound A results in highly significant prevention of the disease. The strong effect we see on body weight and colon inflammation does not correlate with the lymphocyte numbers alone. The synergistic effect is not exclusive to lymphocyte numbers but may be affecting lymphocyte activity or function as well as other cells involved in the disease. These data confirm that a combination of pimecrolimus and Compound A act synergistically in the SCID-IBD mouse model.

### Example 2

Preparation of a Tablet Comprising Compound A and Pimecrolimus

Per tablet having a total weight of ca. 750 mg of a solid dispersion of 30 mg of pimecrolimus and a solid dispersion of 1.00 mg Compound A with 160 mg of hydroxypropylmethylcellulose (3 cps) as a carrier are mixed with 8.90 g of lactose (200 mesh), 0.10 mg of butylated hydroxytoluene, 381.25 mg of lactose spray dried, 150.00 mg of crospovidone, 11.25 mg of silicium dioxide colloidal (Aerosil 200®) and 7.50 mg of magnesium stearate are mixed, the mixture obtained is subjected to dry granulation and the granulated mixture obtained is compressed into tablets of 750 mg (+/−10%) of total weight. A tablet for oral use is obtained.

### Example 3

Utility of a Compound of Formula II in Treating Diseases and Conditions as Specified herein

A) In Vivo: Activity after Topical Administration in Model of Allergic or Allergen-Induced Contact Dermatitis

A1) Oxazolone Allergy (Mouse):

10 μl of a 2% oxazolone solution are applied onto the abdominal skin of mice for sensitization. 8 days later a second exposure with 10 μl of a 2% oxazolone solution is performed by application on the peripheral internal surface of the pinna. 20 minutes and 2 hours after the second exposure has released the challenge reaction, the test solution is applied at the site of the second exposure. Evaluation of the inhibition of inflammation with the test substance is effected by reference to an untreated group treated with the solvent used for dissolving the test substance, alone. 24 hours after the second exposure the animals are killed and the separated pinnae are weighted. The difference in weight between the two pinnae is used for evaluation; the individual differences in the test group and in the solvent control group are statistically compared (by simple variance analysis with subsequent Dunnet Test by normal distribution test if normally distributed, otherwise by Kruskal-Wallis U-test and Wilcoxon-Mann-Witney U-test).

A2) Dintrofluorobenzene (DNFB) Allergy (Swine):

The use of DNFB or dinitrochlorobenzene (DNCB) for inducing a contact allergy is a classical experimental approach which is also being used in humans (P. S. Friedmann and C. Moss, Models in dermatology, 1987, Maibach, Lowe, Ed., Vol. 2, p. 275-281, Karger-Basel). In view of the resemblance between porcine and human skin a corresponding model for topical testing of substances is built up in the swine. on the 1^{st} and 3^{rd} day 100 μl each of a 10% DNFB preparation is applied to the inner surface of the right and, respectively, left thigh. On the 14^{th} day each swine is marled on the right and the left side of the back with circular markings of 5 cm in diameter (8 markings per animal) and 150 μl each of a 5% DNFB preparation is applied thereon. The substances are tested either in the form of galenic compositions or of a solution. The carriers are used in each case as placebo controls. The test products are carefully applied 4 times (first 20 minutes, then 6, 24 and 32 hours after release of challenge reaction). Prior to each application the test areas are evaluated with respect to reddening, swelling and consistence. The coloration of the test areas is then determined quantitatively with a reflectometer, repeatedly. From the data of brightness (L*) and saturation (C*) the erythema index is computed according to the following formula: 100−L*×C*. The mean erythema index is a reflection of the activity according to the following formula:

$$\% inhibition ( 24 , 32 , 48 , 56 hours ) = \frac{delta ( placebo ) - delta ( test )}{delta ( placebo )}$$

$$delta = difference to initial value$$

B) In Vivo: Activity after Topical Administration in the Irritant-Induced Dermatitis Model (Mouse):
B1) 12-O-tetradecanoylphorbol-13-acetate/TPA)-Induced Dermatitis:

Irritation with TPA in test animals is a method for testing substances as to their anti-inflammatory activity after local application (Maibach, Lowe, Ed. Models in Dermatology, Vol. 3, 1987, p. 86-92, Karger-Basel). NMRI mice are given 10 μl of a TPA solution on the inner and outer side of the right pinna (2×10 μl/mouse=2×0,5 μl TPA/mouse). The left pinnae remain untreated. Treatment is effected 30 minutes after irritation, by application of 2×μl of test solution onto the irritated ear surfaces, as described above. The evaluation of the test group is performed by comparison with a group where the right pinna has been treated with only the irritating solution and with the solvent used for the test substance. 6 hours after application of the irritant the animals are killed, the pinnae separated and weighed. The difference in weight of the two pinnae is used for the evaluation, whereby the individual differences of the test groups are statistically compared with the individual differences of the control groups (as under A1).

B2) Dermatitis Induced by Croton Oil

Croton oil may be used, as TPA, in order to induce an irritant-induced dermatitis on which substances can be tested for their anti-inflammatory activity (Maibach, Lowe, Ed., Model in Dermatology, Vol. 3, 1987, p. 86-92, Karger-Basel). NMRI-mice are given 15 μl of 0.23% croton oil (in a mixture of dimethylacetamide, acetone and ethanol 2/4/4) on the inner side of the right pinna. Treatment is effected simultaneously with the irritation, the test substance being dissolved in the solution of irritant applied at the auricular test site. Evaluation of the test group is performed by comparison of the inflammation with a group receiving only the irritant solution on the pinna. The animals are killed 6 hours after application of the irritant, the pinnae separated and weighted. The difference between the weights of the two individual pinnae is used for evaluation by statistical comparison of the single differences in the test group with the single differences in the control group (as under A1).

C) In Vivo: Atopic Dermatitis (Human):

In a randomized, double-blind study, 7 to 12 patients (in total) with moderate to severe atopic dermatitis are treated twice daily for 3 weeks with a composition comprising 0.05 to 2% by weight of a compound of formula II (based on the total weight of the composition) over a defined. symptomatic area of 200 to 1000 cm² of skin. The change in the summary score for erythema, odema and pruritus between first and last days of treatment is evaluated. Local tolerability of study medications and routine safety parameters, including haematology and clinical chemistry, are recorded.

1. Pharmaceutical composition comprising in combination rapamycin or a rapamycin derivative and a compound of formula 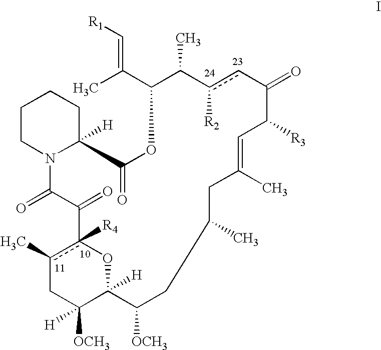
wherein either
R₁ is a group (a) of formula 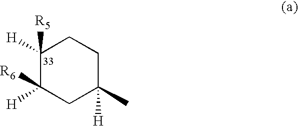
wherein
R₅ is chloro, bromo, iodo or azido,
R₆ is hydroxy or methoxy, and
R₄ is hydroxy and there is a single bond in 10,11 position; or absent, and there is a double bond in 10,11 position
or
R₁ is a group (b) or (c) of formula 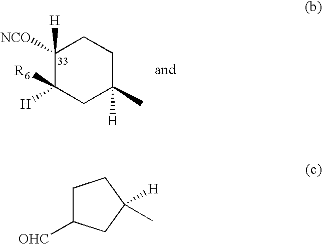
wherein
R₆ is as defined above, and
R₄ is hydroxy and there is a single bond in 10,11 position,
R₂ is oxo and there is a single bond in 23,24 position; optionally protected hydroxy and there is a single or double bond in 23,24 position; or absent and there is a double bond in 23,24 position;
R₃ is methyl, ethyl, propyl or allyl.
beside at least one pharmaceutically acceptable excipient.

2. Pharmaceutical composition of claim 1 wherein a compound of formula I is a compound of formula

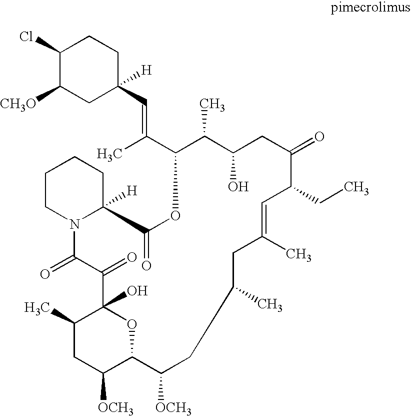

3. Pharmaceutical composition of any one of claims 1 or 2, wherein the rapamycin derivative is a compound of formula 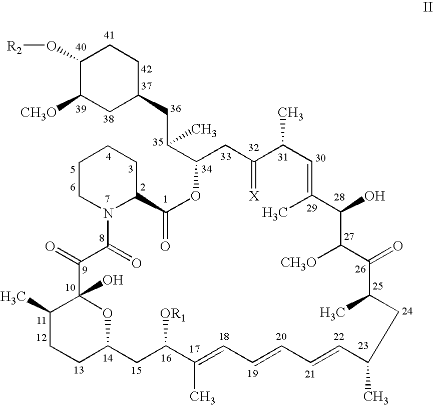
wherein
R₁ is methyl or (C₃₋₆)alkynyl,
R₂ is H, —CH₂—CH₂—OH, 3-hydroxy-2-(hydroxymethyl)-2-methyl-propanoyl or tetrazolyl,
X is =O, (H,H) or (H,OH),
provided that R₂ is other than H when X is =O and R₁ is CH₃,
or a prodrug thereof when R₂ is —CH₂—CH₂—OH.

4. Pharmaceutical composition of any one of claims 1 to 3 comprising in combination 40-O-(2-hydroxyethyl)-rapamycin and pimecrolimus.

5. Pharmaceutical composition according to any one of claims 1 to 4 in the form of a solid dispersion comprising in combination rapamycin or a rapamycin derivative, a compound of formula I and a carrier.

6. Pharmaceutical kit of parts comprising in combination rapamycin or a rapamycin derivative and a compound of formula I beside instructions for simultaneous or sequential administration.

7. Use of a combination of rapamycin or a rapamycin derivative and a compound of formula I as a pharmaceutical.

8. Use of a combination of rapamycin or a rapamycin derivative and a compound of formula I for the manufacture of a medicament for the treatment of inflammatory bowel disease and diseases associated therewith.

9. Use of a combination of rapamycin or a rapamycin derivative and a compound of formula I for the manufacture of a medicament for the treatment of a disease selected from the group consisting of atopic dermatitis, contact dermatitis and further eczematous dermatoses, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias and acne.

10. A method for treatment of an inflammatory disease or an immunologically-mediated disease, including an autoimmune disease, which treatment comprises administering to a subject in need of such treatment an effective amount of a pharmaceutical composition of any one of claims 1 to 5 or of a pharmaceutical kit of parts of claim 6.

11. A method of claim 10, wherein the disease is selected from the group consisting of atopic dermatitis, contact dermatitis and further eczematous dermatoses, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias and acne.

12. A method of claim 10, wherein the disease is inflammatory bowel disease and diseases associated therewith.

13. Use of a combination of rapamycin or a rapamycin derivative and a compound of formula 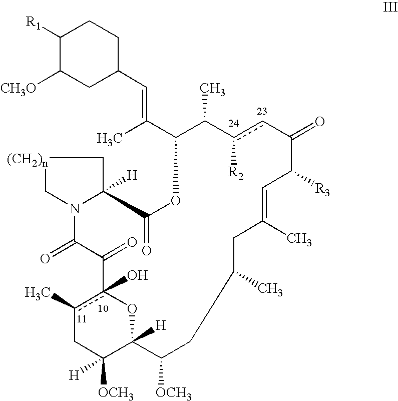
wherein
R₁ is hydroxy or protected hydroxy,
R₂ is hydrogen, hydroxyl or protected hydroxyl,
R₃ is methyl, ethyl, propyl or allyl,
n is an integer of 1 or 2, and
the symbol of a line and dotted line is a single bond for the manufacture of a medicament for the treatment of a disease selected from the group consisting of atopic dermatitis, contact dermatitis and further eczematous dermatoses, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias and acne.

14. Use of a combination of rapamycin or a rapamycin derivative and a compound of formula III for the manufacture of a medicament for the treatment of inflammatory bowel disease and diseases associated therewith.

15. Use of any one of claims 13 or 14 wherein a compound of formula III is a compound of formula

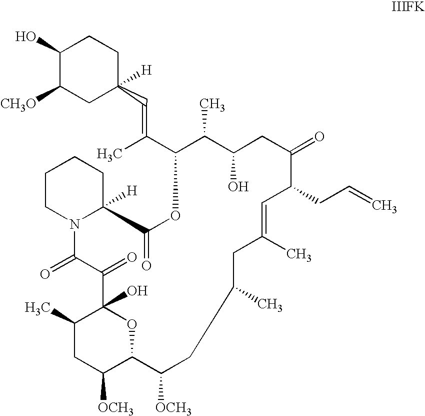

16. Use of a compound of formula 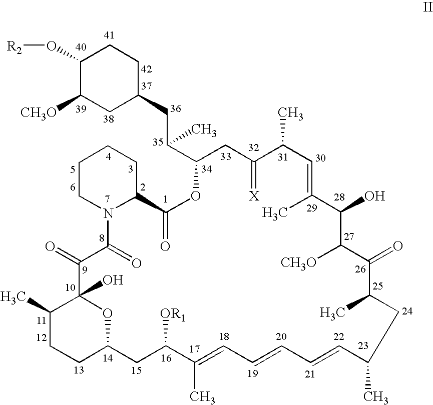
wherein
R₁ is methyl or (C₃₋₆)alkynyl,
R₂ is H, —CH₂—CH₂—OH, 3-hydroxy-2-(hydroxymethyl)-2-methyl-propanoyl or tetrazolyl,
X is =O, (H,H) or (H,OH),
provided that R₂ is other than H when X is =O and R₁ is CH₃,
or a prodrug thereof when R₂ is —CH₂—CH₂—OH for the manufacture of a medicament for the treatment of atopic dermatitis, contact dermatitis and further eczematous dermatoses, seborrhoeic dermatitis, contact hypersensitivity, Lichen planus, Pemphigus, bullous Pemphigoid, Epidermolysis bullosa, urticaria, angioedemas, vasculitides, erythemas, cutaneous eosinophilias and acne.

17. Use of claim 16, wherein the compound of formula II is 40-O-(2-hydroxyethyl)-rapamycin.
